(19) United States
(12) Patent Application Publication
(10) Pub. No.: US 2023/0067142 A1
(43) Pub. Date: Mar. 2, 2023

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Yudai YAMAZAKI, Nasushiobara (JP); Kazumasa NORO, Shioya-gun (JP); Yuka SHIMOMURA, Nasushiobara (JP); Yohei MURAGUCHI, Yaita (JP)

(73) Assignee: CANON MEDICAL SYSTEMS CORPORATION, Otawara-shi (JP)

(21) Appl. No.: 17/820,610

(22) Filed: Aug. 18, 2022

(30) Foreign Application Priority Data
Aug. 27, 2021 (JP) 2021-138908

(51) Int. Cl. G16H 50/50; G16H 50/30

(52) U.S. Cl. CPC G16H 50/50; G16H 50/30

(57) ABSTRACT

A medical information processing device of an embodiment includes a processing circuitry. The processing circuitry acquires clinical data on a subject and a predetermined medical treatment support model, inputs the clinical data to the predetermined medical treatment support model to derive support information regarding a predetermined medical treatment, derives a suitability between the clinical data and the predetermined medical treatment support model on the basis of the clinical data and the predetermined medical treatment support model, and displays the support information and the suitability in association with each other.

### CROSS-REFERENCE TO RELATED APPLICATION

The present application claims priority based on Japanese Patent Application No. 2021-138908 filed Aug. 27, 2021, the content of which is incorporated herein by reference.

### FIELD

Embodiments of the present invention relate to a medical information processing device, a medical information processing method, and a storage medium.

### BACKGROUND

Conventionally, a medical treatment support system that presents support information for supporting a doctor's diagnosis from medical examination results of a patient is known. Specifically, a system for searching for patients with high similarity from past medical treatment data using data related to a stroke, a system for calculating the risk of readmission for heart failure using characteristic information based on diagnosis results of patients and a trained model, and a system for inferring diagnosis names from medical images and outputting one with a reliability of inference results equal to or greater than a threshold value are known. However, these systems are intended for support of doctors of specific diseases and medical departments and do not consider support of doctors of other specialized medical departments or general medical departments. Therefore, when these systems are applied to patients in other medical departments or general medical treatment, there is a possibility that the risk of disease will be calculated to be high or a patient will be selected with a disease even if the patient does not have the disease. This is because a suitability between characteristic information obtained from clinical data for each target patient and a medical treatment model is not taken into account, and thus the same determination result is obtained regardless of the suitability. Therefore, users such as medical professionals are likely to perform erroneous determination from the aforementioned determination results.

### BRIEF DESCRIPTION OF THE DRAWINGS

FIG. 1 is a diagram showing a configuration example of a medical information processing device according to an embodiment.

FIG. 2 is a diagram showing a support information derivation function.

FIG. 3 is a diagram showing derivation of a suitability to a medical treatment support model when there is a single feature vector of clinical data.

FIG. 4 is a diagram showing derivation of a suitability to the medical treatment support model when there are a plurality of feature vectors of clinical data.

FIG. 5 is a diagram showing an example of details of provision support information generated by a provision support information generation function.

FIG. 6 is a diagram showing an example of an image generated by an image generation function.

FIG. 7 is a diagram showing a relationship between the medical treatment support model and a suitability when a target subject derives support information and the suitability.

FIG. 8 is a diagram showing an example of derivation results of similarity.

FIG. 9 is a diagram showing an example of an image showing results of similar cases.

FIG. 10 is a diagram showing an example of a relationship between additional examination items and predicted values of a risk and a suitability when added.

FIG. 11 is a diagram showing an example of an image including information on additional examination items predicted to have a high suitability.

FIG. 12 is a diagram showing details of determination of a disease for a subject.

FIG. 13 is a flowchart showing an example of processing executed by the medical information processing device according to an embodiment.

### DETAILED DESCRIPTION

Hereinafter, a medical information processing device, a medical information processing method, and a storage medium of an embodiment will be described with reference to the drawings.

The medical information processing device of the embodiment includes a processing circuitry. The processing circuitry acquires medical information regarding a subject and a predetermined medical treatment support model, inputs the clinical data to the predetermined medical treatment support model, derives support information regarding predetermined medical treatment, derives a suitability between the clinical data and the predetermined medical treatment support model on the basis of the clinical data and the predetermined medical treatment support model, and displays the support information and the suitability in association with each other.

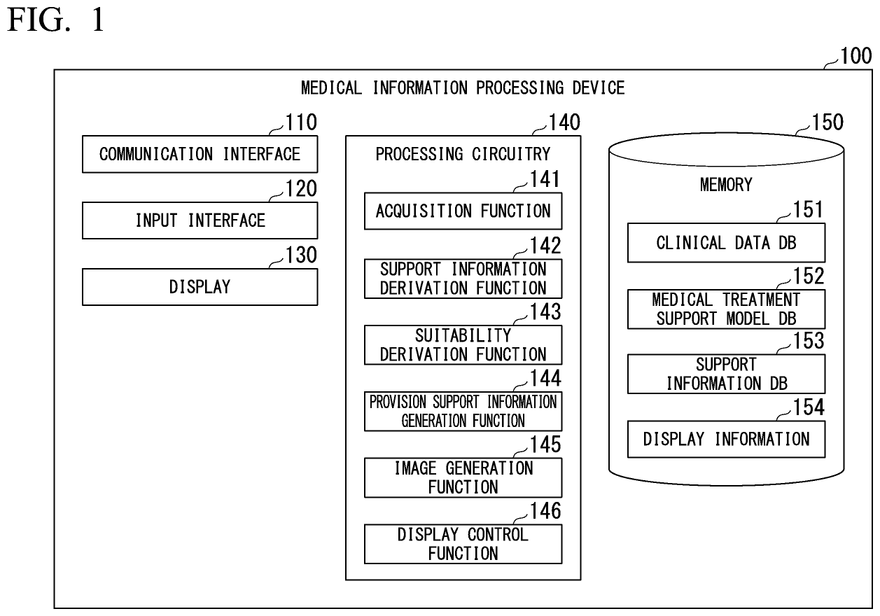

FIG. 1 is a diagram showing an example of a medical information processing device 100 according to an embodiment. The medical information processing device 100 is realized by one or a plurality of processors. For example, the medical information processing device 100 may be a computer included in a cloud computing system or may be a computer (stand-alone computer) that operates independently without depending on other devices.

The medical information processing device 100 includes, for example, a communication interface 110, an input interface 120, a display 130, a processing circuitry 140, and a memory 150.

The communication interface 110 includes, for example, a communication interface such as a network interface card (NIC). The communication interface 110 communicates with external devices via a communication network NW and receives information such as clinical data regarding a subject such as a patient from external devices. External devices are, for example, a medical image generation device, a clinical data server, and a terminal device of each medical department. The medical image generation device is, for example, a device (modality) for capturing a medical image of a subject. Medical image generation devices may include, for example, medical diagnostic devices such as X-ray CT devices and MRI devices, positron emission tomography (PET) devices, PET-computed tomography (CT) devices, single photon emission computed tomography (SPECT) devices, angiography devices, and the like. Medical images generated by medical image generation devices include, for example, images of organs such as the brain, heart, and lungs, or target tissues (target site) such as limbs. The clinical data server is a management device that collects and manages clinical data from examination by doctors and the like in a plurality of medical departments. Further, the clinical data server includes a storage device such as a database, and clinical data is stored in the storage device and managed. Further, the clinical data server may store and manage one or more medical treatment support models.

The communication interface 110 outputs received information to the processing circuitry 140. Further, the communication interface 110 may transmit information to another device connected via the communication network NW under the control of the processing circuitry 140. The other device is, for example, a clinical data server or a terminal device that can be used by doctors, nurses, and the like who use diagnostic information.

The input interface 120 receives various input operations from users such as doctors, converts the received input operations into electrical signals, and outputs the received input operations to the processing circuitry 140. For example, the input interface 120 is realized by a mouse, a keyboard, a trackball, a switch, a button, a joystick, a touch panel, or the like. Further, the input interface 120 may be realized by, for example, a user interface that receives voice input such as a microphone. When the input interface 120 is a touch panel, the display 130 may be formed integrally with the input interface 120.

The display 130 displays various types of information. For example, the display 130 displays an image or the like showing the contents processed by the processing circuitry 140 or displays a graphical user interface (GUI) or the like for receiving various input operations from a user. For example, the display 130 is a liquid crystal display (LCD), a cathode ray tube (CRT) display, an organic electroluminescence (EL) display, or the like. Further, the display 130 may include a speaker or the like that performs voice output and may output vocal sound, a warning sound, or the like associated with a displayed image according to the control of the processing circuitry 140.

The processing circuitry 140 includes, for example, an acquisition function 141, a support information derivation function 142, a suitability derivation function 143, a provision support information generation function 144, an image generation function 145, and a display control function 146. The acquisition function 141 is an example of an “acquisition unit.” The suitability derivation function 143 is an example of a “suitability derivation unit.” The support information derivation function 142 is an example of a “support information derivation unit.” The provision support information generation function 144 is an example of a “providing support information generation unit.” The image generation function 145 is an example of an “image generation unit.” The display control function 146 is an example of a “display control unit.” The processing circuitry 140 realizes these functions by, for example, a hardware processor executing a program stored in a memory (storage device or storage circuit) 150.

The hardware processor refers to, for example, a circuit (circuitry) such as a central processing unit (CPU), a graphics processing unit (GPU), an application specific integrated circuit (ASIC), or a programmable logic device (for example, a simple programmable logic device (SPLD) or a complex programmable logic device (CPLD), or a field programmable gate array (FPGA)). Instead of storing the program in the memory 150, the program may be configured to be directly embedded in the circuit of the hardware processor. In this case, the hardware processor realizes the functions by reading and executing the program embedded in the circuit. The aforementioned program may be stored in the memory 150 in advance, or may be stored in a non-temporary storage medium such as a DVD or a CD-ROM and installed in the memory 150 from the non-temporary storage medium when the non-temporary storage medium is set in a drive device (not shown) of the medical information processing device 100. The hardware processor is not limited to one configured as a single circuit, and may be configured as one hardware processor by combining a plurality of independent circuits to realize each function. A plurality of components may be integrated into one hardware processor to realize each function.

Each component of the processing circuitry 140 may be decentralized and realized by a plurality of hardware devices. The processing circuitry 140 may be realized by a processing device capable of communicating with the medical information processing device 100, instead of a component of the medical information processing device 100. The processing device is, for example, a workstation connected to a single medical information processing device 100 or a device (e.g., a cloud server) that is connected to a plurality of medical information processing devices 100 and collectively executes the same processing as that performed by the processing circuitry 140 which will be described below.

For example, the acquisition function 141 acquires clinical data on an arbitrary subject from an external device via the communication interface 110. The clinical data includes, for example, subject information (identification information for identifying the subject, attribute information, biological information, and the like), opinion information of a diagnostician such as a doctor, and information such as a treatment history of the subject. The identification information includes, for example, an identification ID (for example, a patient ID or a subject ID), an address, a name, and the like. The attribute information includes, for example, height, weight, age, sex, and the like. The biological information includes, for example, a pulse, a heart rate, a respiratory rate, a blood pressure, a body temperature and the like. The treatment history includes, for example, information such as current medical conditions, medical history, drugs, and treatment details. In addition, the clinical data may include medical images generated by a medical image generation device and imaging conditions of medical images (for example, date and time, an imaging site, an imaging device, an imaging method (scan conditions), and reconstruction conditions). The information (items) included in the clinical data is not limited to the above-mentioned example, and may include other items that can be obtained at the time of medical treatment of the subject. The acquisition function 141 stores the acquired clinical data in a clinical data database (DB) 151 in the memory 150.

Further, the acquisition function 141 acquires a medical treatment support model associated with a predetermined medical treatment from an external device or the like via the communication interface 110, for example. The details of the medical treatment support model will be described later. The acquisition function 141 stores the acquired medical treatment support model in a medical treatment support model DB 152 in the memory 150.

The support information derivation function 142 inputs clinical data to the medical treatment support model to derive support information regarding a predetermined medical treatment on the basis of the clinical data acquired by the acquisition function 141 and the medical treatment support model. The support information regarding a predetermined medical treatment includes, for example, risk information regarding a disease. The risk information includes an onset (disease) risk indicating the probability (possibility) of developing (or having) a predetermined disease, an exacerbation risk indicating the probability that the disease worsens, an aggravation risk indicating the probability that the disease becomes severe, a hospitalization risk indicating the probability of hospitalization within a predetermined period from the present, a readmission risk indicating the probability of readmission within a predetermined period after discharge, and the like. In addition, the support information derivation function 142 may derive risk information indicating a negative probability (for example, probability of non-onset (or non-having), probability of non-deterioration) instead of (or in addition to) the positive probability described above. The support information derivation function 142 derives risk information for each disease type, for example. The details of the support information derivation function 142 will be described later.

The suitability derivation function 143 derives a suitability between the clinical data acquired by the acquisition function 141 and the medical treatment support model. A suitability is, for example, an index value indicating to what extent items included in clinical data are suitable as input data (input elements) with respect to the medical treatment support model (for example, whether the items are suitable for input data or how many necessary items are included in the input data) after one or more symptoms are predicted by the medical treatment support model. The details of the suitability derivation function 143 will be described later.

The provision support information generation function 144 generates provision support information to be provided (for example, to be output through the display 130) to a user on the basis of the suitability derived by the suitability derivation function 143 from the support information acquired by the acquisition function 141. For example, the provision support information generation function 144 generates support information (support information with suitability) having a suitability associated with the support information acquired by the acquisition function 141 as the provision support information. The details of the provision support information generation function 144 will be described later.

The image generation function 145 generates an image including support information regarding a predetermined medical treatment to be provided to the user on the basis of the provision support information generated by the provision support information generation function 144. The details of the image generation function 145 will be described later.

The display control function 146 causes the display 130 or the like to display the image or the like generated by the image generation function 145. Further, the display control function 146 may cause the display 130 or the like to display images showing each piece of information acquired by the acquisition function 141, information derived by the suitability derivation function 143, information derived by the support information derivation function 142, and the like. Further, the display control function 146 may store the information to be displayed in the memory 150 as display information 154, or may transmit the information to the clinical data server or other devices via the communication network NW. Further, the display control function 146 may generate vocal sound, a warning sound, or the like corresponding to the information to be displayed and output it through a speaker or the like. The display control function 146 may execute processing executed by the above-described provision support information generation function and the image generation function 145.

The memory 150 is realized by, for example, a semiconductor memory element such as a random access memory (RAM) or a flash memory, a hard disk, an optical disc, or the like. These non-transient storage media may be realized by other storage devices connected via the communication network NW, such as a network attached storage (NAS) and an external storage server device. Further, the memory 150 may include a transient storage medium such as a read only memory (ROM) or a register. The memory 150 stores, for example, the clinical data DB 151, the medical treatment support model DB 152, a support information DB 153, display information 154, a program, and various other types of information.

Here, the medical treatment support model stored in the medical treatment support model DB 152 is a trained model that is trained by machine learning, deep learning, artificial intelligence (AI), or the like using a teacher data group, for example. Teacher data is, for example, data in which a correct answer medical treatment result is associated with input data based on each item included in clinical data. For example, the medical treatment support model may be a model having information of at least one item included in clinical data as an input and having a probability of developing a predetermined disease (and/or a probability of not developing a predetermined disease) as an output. The predetermined disease includes, for example, at least one of heart failure, lung cancer, breast cancer, COVID-19, diabetes, cerebral infarction, and the like. The medical treatment support model DB 152 may store a medical treatment support model for each disease type or may store medical treatment support models corresponding to a plurality of diseases. Further, the medical treatment support model may be, for example, a model associated with the same disease or a model in which input data and output data items are adjusted according to a medical treatment purpose or a degree of medical treatment. Accordingly, it is possible to obtain different types of support information (diagnosis results) in a case where a medical treatment purpose of a subject is a regular medical examination and in a case where the medical treatment purpose is re-examination as a result of the regular medical examination.

Further, the medical treatment support model stored in the medical treatment support model DB 152 may include an identification model and a generation model. The identification model is, for example, a model in which the boundaries of classes (groups) are trained. In the identification model, an objective function (discriminant function) that combines the attributes of correct answer medical treatment results (for example, a probability of developing a predetermined disease and a probability of not developing a predetermined disease) is generated and unknown data is classified into predetermined attributes on the basis of the generated function and the like at the time of classifying medical treatment results from input data by supervised learning, for example. The generation model is a model in which a distribution of data in each class is trained. The generation model sets a probability distribution that generates information corresponding to a correct answer medical treatment result and generates a medical treatment result from unknown data on the basis of the probability distribution.

Further, the medical treatment support model stored in the medical treatment support model DB 152 may include a linear model and a non-linear model. In the case of a linear model, data is classified (identified) by a straight line (linear function) or a plane, and numerical values are predicted (generated). In the case of a non-linear model, data is classified (identified)using curves and curved surfaces, and numerical values are predicted (generated).

The support information DB 153 stores support information derived by the support information derivation function 142 and provision support information (for example, support information with suitability) generated by the provision support information generation function 144. Further, the support information DB 153 may store past support information (history information) for the same subject. Further, the support information DB 153 may store information regarding a suitability derived by the suitability derivation function 143.

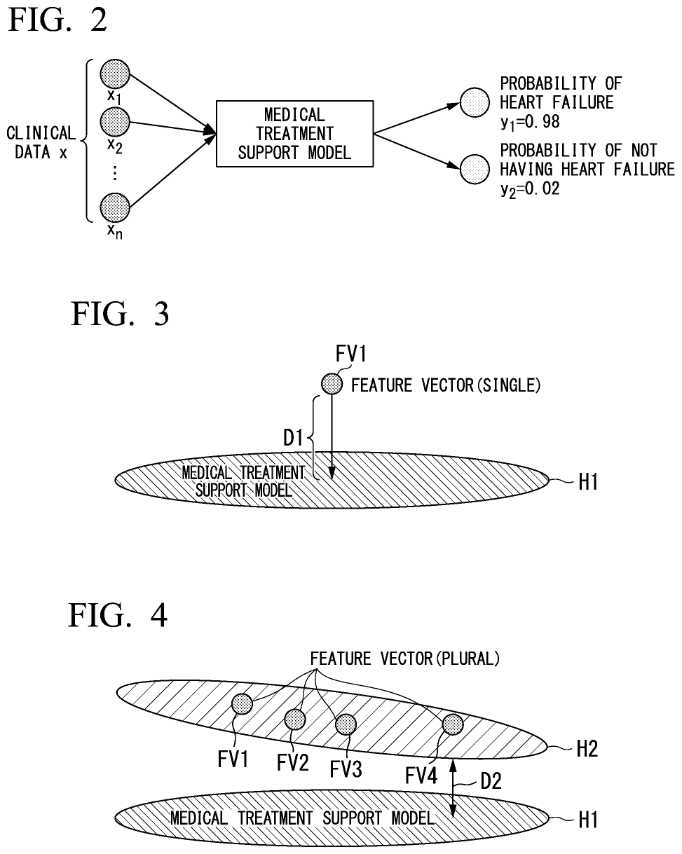

Next, the details of the support information derivation function 142 will be described. FIG. 2 is a diagram showing the support information derivation function 142. A medical treatment support model shown in FIG. 2 is one of the models stored in the medical treatment support model DB 152 and is a model for deriving the risk of developing heart failure. For example, the support information derivation function 142 inputs input data (x₁, x₂, . . . xₙ) corresponding to a model included in clinical data x to the medical treatment support model shown in FIG. 2 and derives support information (y=[y₁, y₂]^{T}) including a probability y₁ of having heart failure and a probability y₂ of not having heart failure. Further, the support information derivation function 142 may input, for example, clinical data (time-series clinical data) acquired at different times for the same subject to the medical treatment support model and derive support information (y={yᵢ}ᵢ₌₀^{N}(yᵢ=[y₁, y₂] ^{T})). In addition, the support information derivation function 142 may derive support information including a probability of a subject associated with clinical data having another disease (and/or a probability of the subject not having another disease) using a model for deriving the risk of other diseases stored in the medical treatment support model DB 152. Further, the support information derivation function 142 may derive support information for a subject associated with clinical data using a model having the clinical data x as input and having similarities of symptoms to other subjects (disease patients) as output.

The support information derivation function 142 selects, for example, a medical treatment support model to be used from the medical treatment support model DB 152 on the basis of setting information input from a user in advance through the input interface 120 and derives support information. In this case, the image generation function 145 generates images showing information about one or a plurality of medical treatment support models stored in the medical treatment support model DB 152 and causes the display 130 to display the images through the display control function 146. The user inputs a medical treatment support model to be used through the input interface 120 among medical treatment support models displayed on the display 130. Accordingly, the support information derivation function 142 can acquire information input from the input interface 120 as setting information. When the user specifies a medical treatment model for diagnosing a disease which is not stored in the medical treatment support model DB 152, the support information derivation function 142 acquires the corresponding medical treatment support model from an external device via the communication interface 110.

In addition, the support information derivation function 142 may select a medical treatment support model in which a suitability derived by the suitability derivation function 143 is predicted to be equal to or higher than a threshold value and derive support information on the basis of input data (input items) included in the support information x instead of (or in addition to) selecting a medical treatment support model on the basis of setting information from the user. Further, the support information derivation function 142 may select a medical treatment support model on the basis of a medical treatment purpose, subject information, opinion information, and the like. For example, when a subject is a female, the support information derivation function 142 derives support information using a medical treatment support model for driving the risk of developing breast cancer or derives support information using a medical treatment support model associated with diseases that are more likely to develop depending on age. Accordingly, it is possible to efficiently derive support information using a more appropriate model.

Instead of (or in addition) selecting a medical treatment support model, the support information derivation function 142 may select items input to a medical treatment support model associated with a disease to be derived from items included in clinical data. In this case, the support information derivation function 142 may select input items on the basis of setting information input from the user in advance through the input interface 120 or select input items on the basis of a medical treatment purpose, subject information, opinion information, and the like. Accordingly, items to be input to a medical treatment support model corresponding to a disease to be derived can be adjusted according to the user and the purpose and use of a medical examination.

The support information derivation function 142 stores the derived support information in the support information DB 153 of the memory 150.

Next, the details of the suitability derivation function 143 will be described. For example, the suitability derivation function 143 derives suitability between input items (input elements) of a medical treatment support model used in the support information derivation function 142 and items (examination items and the like) included in clinical data. The suitability derivation function 143 derives the degree of suitability (suitability) of a model of clinical data on the basis of, for example, a medical treatment support model, information on an objective function (loss function) at the time of model learning, and clinical data acquired by the acquisition function 141. For example, the suitability derivation function 143 makes a method of deriving a suitability to a clinical data model different in a case where there is a single feature vector, which is an example of feature information based on the clinical data, and a case where there are a plurality of feature vectors. A feature vector is a vector in which the content of items included in clinical data is used as a feature amount and this feature amount is represented in a vector format. The feature vector is represented, for example, as one point in an N-dimensional vector space. Each axis of N dimension is, for example, an item (for example, an examination item, an attribute of a subject, or the like) associated with a predetermined disease. Hereinafter, a case where there is a single feature vector and a case where there are a plurality of feature vectors will be described.

(Case where there is Single Feature Vector)

FIG. 3 is a diagram showing derivation of a suitability to a medical treatment support model in a case where there is a single feature vector of clinical data. The suitability derivation function 143 sets, for example, a pseudo classification label (hereinafter referred to as a “pseudo label”) in support information when a medical treatment support model for which a suitability is derived is an identification model, and derives a likelihood (index value indicating how much the support information is suitable for diagnostic results (correct answer diagnostic results)) on the basis of an objective function (for example, cross entropy error in the case of classification), the support information, and the pseudo label (for example, y=[1, 0]^{T}). Then, the suitability derivation function 143 derives a suitability by normalizing the derived likelihood to 0 to 1 (or 0% to 100%). For example, the suitability derivation function 143 increases the suitability as the likelihood increases. The same applies to the following description.

Further, when the medical treatment support model is a linear generation model (when it is a linear model and is a generation model), the suitability derivation function 143 derives a suitability by normalizing a distance (projection distance) D1 between a feature vector FV1 of clinical data in a vector space and a medical treatment support model (hyperplane H1) to 0 to 1 (or 0% to 100%), as shown in FIG. 3. For example, the suitability derivation function 143 increases the suitability as the distance D1 decreases. Further, when the medical treatment support model is a nonlinear generation model (when it is a nonlinear model and is a generation model), the suitability derivation function 143 derives the aforementioned likelihood when the clinical data corresponding to the feature vector is applied to the medical treatment support model as a suitability. The suitability in this case is normalized to 0 to 1 (or 0% to 100%).

(Case where there are Plurality of Feature Vectors)

FIG. 4 is a diagram showing derivation of a suitability to a medical treatment support model when there are a plurality of feature vectors of clinical data. For example, when the medical treatment support model is an identification model, the suitability derivation function 143 sets a pseudo label (for example, y=[1, 0]^{T}) of support information and derives a likelihood statistic on the basis of an objective function (for example, cross entropy error in the case of classification), the support information, and the pseudo label using the set label. The statistic is, for example, an average value, a maximum value, or a minimum value. Then, the suitability derivation function 143 derives the suitability by normalizing the derived likelihood to 0 to 1 (or 0% to 100%).

In addition, when the medical treatment support model is a linear generation model, the suitability derivation function 143 derives a suitability by normalizing a distance D2 between a subspace (hyperplane H2) that approximates a plurality of feature vectors (FV1 to FV4 in the example of FIG. 4) obtained from a plurality of pieces of clinical data at different times and the medical treatment support model (hyperplane H1) to 0 to 1 (or 0% to 100%), for example, as shown in FIG. 4. For example, the suitability derivation function 143 increases the suitability as the distance D2 decreases. The distance between the subspace (hyperplane H2) and the medical treatment support model (hyperplane H1) may be, for example, a shortest distance, a longest distance, or an average distance. Further, the suitability derivation function 143 derives a suitability by, for example, normalizing an angle (regular angle) formed by the hyperplane H2 and the hyperplane H1 to 0 to 1 (or 0% to 100%). For example, the suitability derivation function 143 increases the suitability as the regular angle decreases.

Further, when the medical treatment support model is a non-linear generative model, the suitability derivation function 143 approximates a plurality of feature vectors (clinical data) with a probability distribution and derives a distance from a probability distribution representing the medical treatment support model as a suitability. The distance of the probability distribution is, for example, a distance of a probability distribution space based on Kullback Leibler (KL) divergence, a divergence, and p divergence. Further, the suitability derivation function 143 normalizes the derived suitability to 0 to 1 (or 0% to 100%).

Next, the details of the provision support information generation function 144 will be described. When there is a risk with respect to one or more diseases in a subject associated with clinical data according to support information derivation function 142, for example, the provision support information generation function 144 generates support information for provision (support information with suitability) in which information on the risk is associated with a suitability for each disease type (for example, disease name).

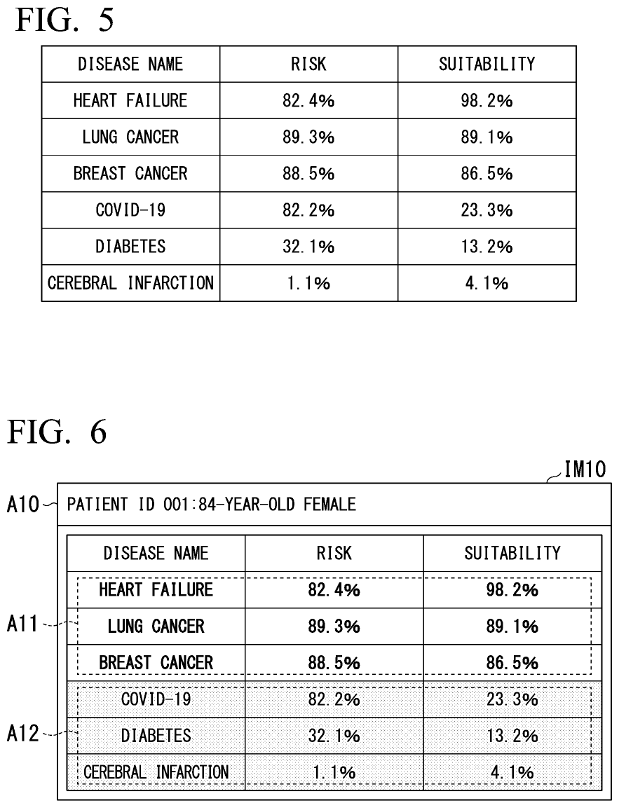

FIG. 5 is a diagram showing an example of details of support information for provision generated by the provision support information generation function 144. The support information for provision shown in FIG. 5 is information in which a risk and a suitability are associated with a disease name. The support information for provision shown in FIG. 5 may be generated for each subject. In addition, although the support information for provision is sorted in descending order of suitability in the example of FIG. 5, the support information for provision may be sorted in descending order of risk. Further, the provision support information generation function 144 may generate support information for provision having a risk equal to or greater than a threshold value or may generate support information for provision having a suitability equal to or greater than a threshold value. The provision support information generation function 144 stores the generated support information for provision in the support information DB 153 of the memory 150.

The image generation function 145 generates an image to be displayed on the display 130 or the like and provided to the user on the basis of the support information for provision generated by the provision support information generation function 144.

FIG. 6 is a diagram showing an example of an image IM10 generated by the image generation function 145. The content, layout, colors, design, and other display modes displayed in the image IM10 which will be described below are not limited to this. The same applies to the description of other images which will be described below.

The image IM10 shown in FIG. 6 includes, for example, a subject information display area A10, a first support information display area A11, and a second support information display area A12. In the subject information display area A10, subject information associated with clinical data is displayed. In the example of FIG. 6, a patient ID which is identification information of a subject, and the age and sex of the subject are displayed in the subject information display area A10. In the first support information display area A11, for example, support information with suitability (disease name, risk, and suitability) having a suitability equal to or greater than a threshold value among the support information for provision is displayed. In the second support information display area A12, for example, support information with suitability having a suitability less than the threshold value among the support information for provision is displayed.

The display control function 146 causes the display 130 to display the image displayed by the image generation function 145. In this case, the display control function 146 may make a display mode of information displayed in the first support information display area A11 different from a display mode of information displayed in the second support information display area A12 and cause the information to be displayed in a distinguishable manner. For example, the display control function 146 performs display for causing the user to easily ascertain that the information displayed in the first support information display area A11 has a higher importance (priority) than the information displayed in the second support information display area A12. For example, the display control function 146 displays the information displayed in the second support information display area A12 in a display mode that is not emphasized. The display mode that is not emphasized is, for example, gray display of characters or a background, display in which a background is transparent to characters with a predetermined transmittance, and the like. Further, the display control function 146 may display the information displayed in the first support information display area A11 with more emphasis than the information displayed in the second support information display area A. Emphasized display means, for example, highlighted display, blinking display, and displaying characters in a highlighted color such as red. Further, the display control function 146 may not display the information in the second support information display area A12 and generate an image showing support information with suitability having a suitability equal to or greater than the threshold value. Accordingly, it is possible to cause the user to easily visually recognize important support information. Meanwhile, which of the above-mentioned display modes will be used for display may be selected according to the number of pieces of support information (for example, the number of diseases) displayed in each area, or selected according to the magnitude of suitability, the magnitude of risk, and a disease type. Further, the display control function 146 may select a display mode on the basis of setting information from the user received through the input interface 120.

### First Modified Example

Next, a first modified example of support information for provision generated by the provision support information generation function 144 will be described. In the first modified example, the provision support information generation function 144 searches for past similar cases of other subjects on the basis of support information with suitability of a target subject and generates search results as support information for provision. In this case, the provision support information generation function 144 applies, for example, a medical treatment support model corresponding to a predetermined disease used in the support information derivation function 142 and the suitability derivation function 143 to past clinical data of the subject to derive support information and a suitability, and derives a similarity to support information of other subjects on the basis of the derivation result.

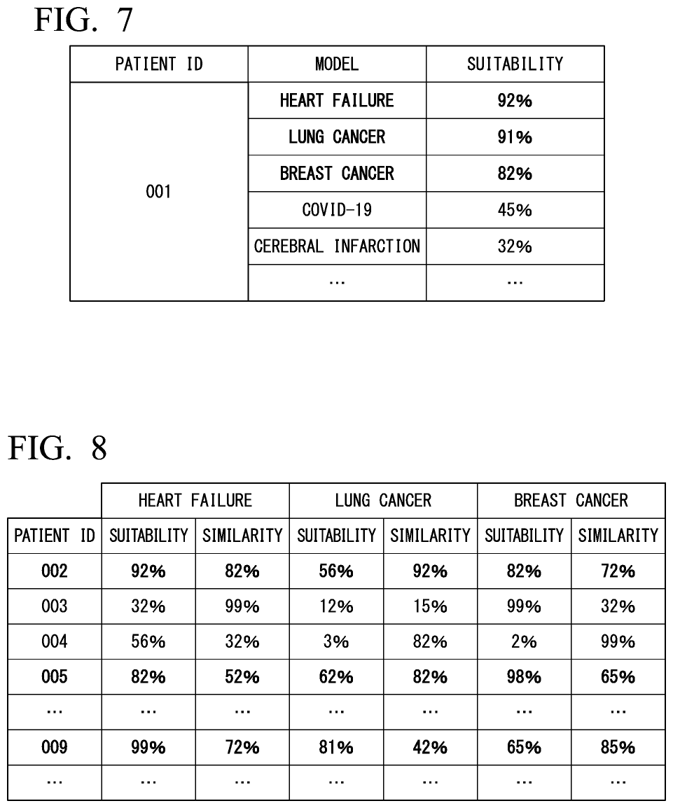

FIG. 7 is a diagram showing a relationship between a medical treatment support model and a suitability when a target subject derives support information and the suitability. The example of FIG. 7 shows a relationship between a model (medical treatment support model) used in the support information derivation function 142 and the suitability derivation function 143 when support information with suitability of the target subject (patient ID: 001) is generated, and a suitability for each model. The provision support information generation function 144 searches for similar cases to other subjects using, for example, a model having a suitability equal to or greater than a threshold value (for example, 80% or higher) among the support information with suitability shown in FIG. 7. In the example of FIG. 7, diseases associated with the medical treatment support model having a suitability of 80% or higher are “heart failure,” “lung cancer,” and “breast cancer.” Therefore, the provision support information generation function 144 extracts a medical treatment support model for diseases of heart failure, lung cancer, and breast cancer from the medical treatment support model DB 152 and causes the support information derivation function 142 and the suitability derivation function 143 to derive support information and a suitability using clinical data of other subjects stored in the clinical data DB 151 as input using the extracted model. Then, the provision support information generation function 144 derives a similarity between a suitability (heart failure: 92%, lung cancer: 91%, breast cancer: 82%) for target disease of the target subject and a suitability of another subject.

FIG. 8 is a diagram showing an example of similarity derivation results. In the example of FIG. 8, a suitability and a similarity for each of other subjects (patients) are represented for each target disease (heart failure, lung cancer, or breast cancer). The information shown in FIG. 8 is an example of support information for provision. For example, the provision support information generation function 144 compares suitabilities of heart failure, lung cancer, and breast cancer of the target subject (patient ID: 001) with suitabilities of heart failure, lung cancer, and breast cancer of another subject, and derives a similarity such that the suitability increases as suitability error decreases.

Further, the provision support information generation function 144 may compare items of clinical data of the target subject with items of clinical data of another subject and derive a similarity such that the similarity increases as error decreases instead of (or in addition to) the above-described derivation method. In this case, the provision support information generation function 144 performs comparison with respect to items that are inputs of a model for each disease among items included in the clinical data to derive a similarity. For example, when a similarity for heart failure is derived, the provision support information generation function 144 derives a similarity on the basis of change in the weight of the subject included in the clinical data, symptoms such as shortness of breath, dullness, and presence or absence of swelling, and a suitability such as whether or not there are various diseases such as myocardial infarction and angina, arteriosclerosis, hypertension, valvular disease, myocardiosis, arrhythmia, and congenital heart disease. In addition, when a similarity for lung cancer or breast cancer is derived, the provision support information generation function 144 derives a similarity for items such as the position, size, and number, and progress of cancer, a treatment method, and age included in the clinical data. The provision support information generation function 144 may derive a similarity for an item determined for each disease in advance or may derive a similarity on the basis of an item input from a user through the input interface 120. In addition, a similarity may be derived on the basis of common items for one or more diseases.

When the similarity is derived, the provision support information generation function 144 generates support information for provision as similar case results including support information (support information for provision) and clinical data of other subjects having similarities equal to or greater than the threshold value. The image generation function 145 generates an image including the generated support information for provision and causes the generated image to be displayed on the display 130 or the like through the display control function 146.

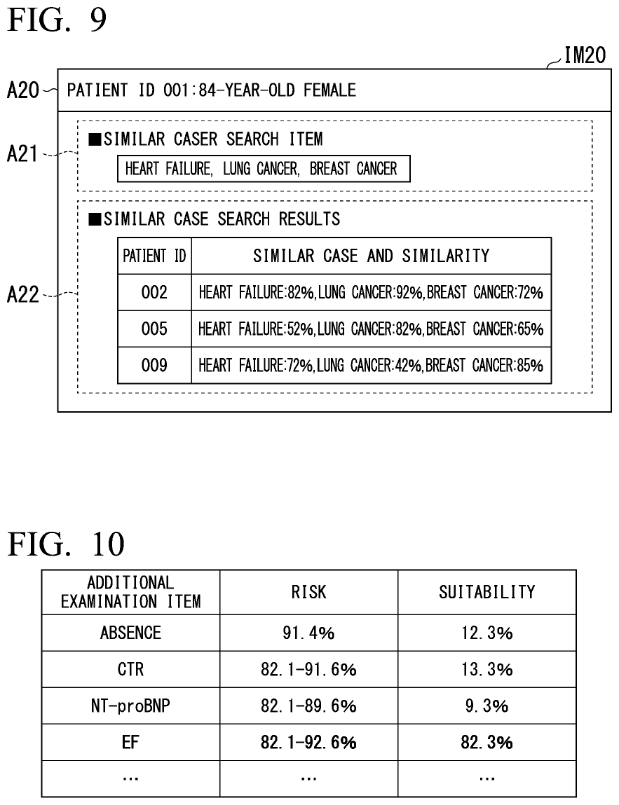

FIG. 9 is a diagram showing an example of an image IM20 showing similar case results. The image IM 20 includes, for example, a subject information display area A20, a search item display area A21, and a search result display area A22. In the subject information display area A20, identification information for identifying a subject is displayed. In the example of FIG. 9, a patient ID, age, and sex of a target subject are displayed in the subject information display area A20.

In the search item display area A21, search items used in similar case search are displayed. Specifically, in the search item display area A21, details of a disease (medical treatment support master) having a suitability of the target subject equal to or greater than the threshold value is displayed. In the search result display area A22. similar cases of other subjects having similarities equal to or greater than a predetermined value and the similarities in heart failure, lung cancer, and breast cancer are displayed as similar case search results. In the example of FIG. 9, the fact that each of patients having patient IDs of 002, 005, and 009 has diseases of heart failure, lung cancer, and breast cancer, and similarities to a target subject (support information and clinical data) with respect to each disease are displayed in the search result display area A22.

Further, although all similarities of diseases included in search items of similar cases are evaluated overall and information with a high similarity (for example, having a small error in a suitability for each disease of the target patient) is displayed as information with a high similarity in the example of FIG. 9, the displayed content is not limited thereto. For example, the image generation function 145 and the display control function 146 may generate and display an image representing a suitability or may generate and display an image representing search results in descending order of similarity for each disease. Further, the image generation function 145 and the display control function 146 may generate and display an image displaying all similar case search results regardless of the degree of similarity.

According to the first modified example described above, it is possible to allow a user to estimate a disease of a target subject by with reference to clinical data of other subjects having cases with high similarities to the target subject and to easily determine the subsequent treatment policy, examination items, and the like.

### Second Modified Example

Next, a second modified example of support information for provision generated by the provision support information generation function 144 will be described. For example, if a medical treatment support model learns a wide range of diseases and various subjects rather than a specific disease, a suitability may be high for any subject, and it is impossible to perform comparison with past subjects according to clinical data of a target subject, and the like, and thus suitability is likely to decrease. Therefore, as the second modified example, the provision support information generation function 144 derives a suitability when data representing examinations that have not been performed on the target subject is added to the clinical data and generates, as support information for provision, information indicating which examination causes the suitability to increase when added (for example, an additional examination item estimated to have a high suitability) on the basis of the derivation result.

For example, the provision support information generation function 144 extracts additional examination items predicted to have a suitability i higher than the threshold value by including additional examination items with current clinical data as x, additional examination item as z, and suitability as q. For example, the provision support information generation function 144 extracts an additional examination item z predicted to have a high suitability according to “suitability i=argmaxᵢ(q(x+zᵢ)).” For example, the provision support information generation function 144 derives how the risk and suitability are different depending on differences in input data (examination items) with respect to a medical treatment support model included in the clinical data on the basis of past clinical data and support information (support information for provision).

FIG. 10 is a diagram showing an example of a relationship between an additional examination item and predicted values of a risk and a suitability when the additional examination item is added. In the example of FIG. 10, a disease risk and a suitability are associated with each additional test item in a case in which a disease is “heart failure.” FIG. 10 also includes a risk and a suitability when there are no additional examination items (when there is no zᵢ). The risk and suitability may be specific ranges rather than specific values. In the example of FIG. 10, cardio thoracic ratio (CTR), NT-proBNP (human brain natriuretic peptide precursor N-end fragment), ejection fraction (EF; ventricular ejection fraction), and the like are represented as additional examination items, and predicted values of a risk and a suitability when each of the additional examination items is added are represented. The provision support information generation function 144 also generates support information that predicts risks and suitabilities for additional examination items for diseases other than heart failure.

The provision support information generation function 144 generates support information as shown in FIG. 10, causes the image generation function 145 to generate an image including information on additional examination items having suitabilities equal to or greater than the threshold value among the generated support information, and causes the display control function 146 to display the generated image in a predetermined display mode.

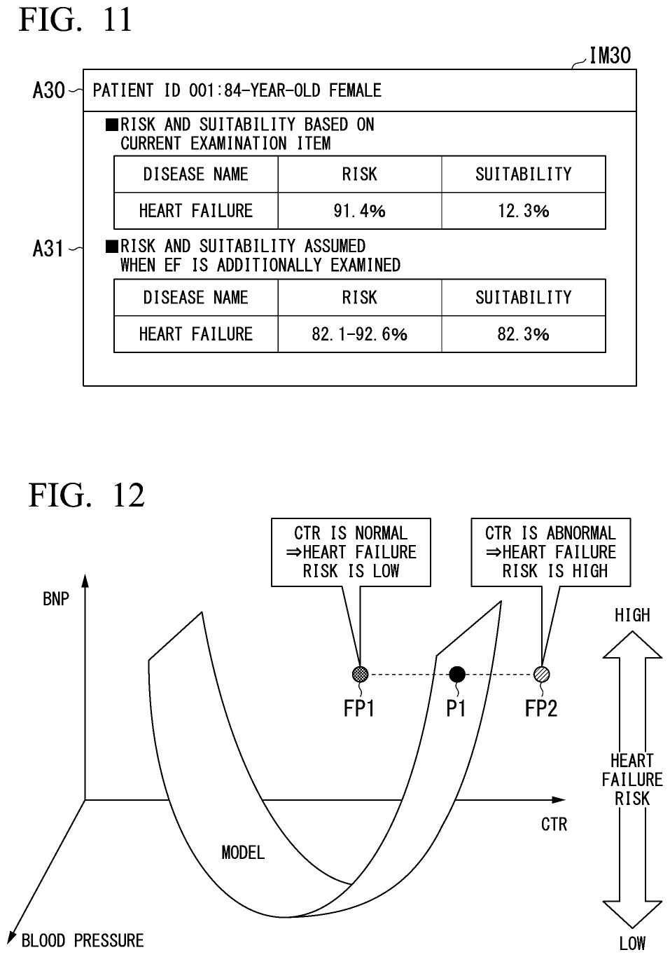

FIG. 11 is a diagram showing an example of an image IM30 including information on an additional examination item predicted to have a high suitability. The image IM30 shown in FIG. 11 includes, for example, a subject information display area A30 and a support information display area A31. In the subject information display area A30, identification information for identifying a subject is displayed. In the example of FIG. 11, a patient ID, which is identification information of a subject, and the age and sex of the subject are displayed in the subject information display area A30.

In the support information display area A31, a disease risk and a suitability based on the current examination item, and predicted values of a disease risk and a suitability assumed when an additional examination item having a suitability equal to or greater than the threshold value (or a maximum suitability) is added for a predetermined disease name (for example, heart failure). The threshold value here is, for example, a value equal to or greater than the suitability based on the current examination item. Accordingly, it is possible to further improve the suitability of support information by adding an additional examination item.

According to the second modified example described above, it is possible to allow a user to easily determine what kind of clinical data (examination item) should be added for a target subject. In addition, it is possible to improve the suitability of support information by adding an additional examination item, and thus it is possible to provide more appropriate support information on a subject having an undetermined or unknown disease to the user. Therefore, it is possible to assist the user in diagnosing the subject more appropriately.

Here, an example of differences in support information between a conventional method and the method according to the embodiment will be described. FIG. 12 is a diagram showing details of determination a disease for a subject. The example of FIG. 12 shows a positional relationship between points FP1 and FP2 of two different subject feature vectors in a vector space with blood pressure, CTR, and brain natriuretic peptide (BNP) as respective axes in three dimensions, and a nonlinear model (a curved surface medical treatment support model) corresponding to heart failure. For example, it is assumed that a risk is determined on the basis of a shortest distance from the feature vectors to the model curved surface, and distances from the points FP1 and FP2 of the feature vectors to a shortest point P1 of the curved surface are identical. Here, CTR is normal and the risk of heart failure is low at point FP1, whereas CTR is abnormal and the risk of heart failure is high at point FP2. Here, since suitability is not taken into account in the conventional method, the risk is determined simply based on the shortest distance, and thus the same risk determination result is obtained.

Therefore, as shown in the above-described embodiment, a suitability of clinical data of a target subject to a medical treatment support model is also derived to cause a difference between suitabilities for the feature vectors of the two subjects (causes the suitability at point FP2 to be higher than that at point FP1), and thus the risk of the subject can be derived more accurately. In addition, according to the embodiment, it is possible to allow the user to determine symptoms of a subject more appropriately by providing support information with suitability as support information for provision.

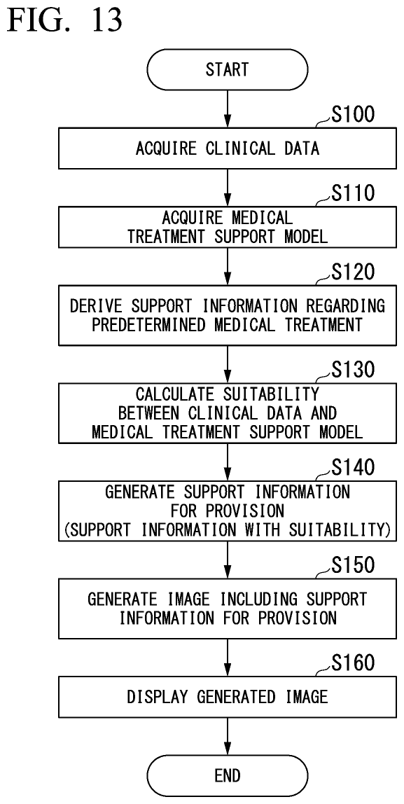

FIG. 13 is a flowchart showing an example of processing executed by the medical information processing device 100 according to the embodiment. In the example of FIG. 13, the acquisition function 141 acquires clinical data from an external device, the clinical data DB 151, or the like (step S100). Further, the acquisition function 141 acquires a medical treatment support model from an external device or the medical treatment support model DB 152 (step S110).

Next, the support information derivation function 142 derives support information related to a predetermined medical treatment (step S120). Next, the suitability derivation function 143 calculates a suitability between the clinical data and the medical treatment support model (step S130). Processing of step S120 and processing of step S130 may be executed in the reverse order. Next, the provision support information generation function 144 generates support information for provision (support information with suitability) by adding the suitability calculated by the suitability derivation function 143 to the support information derived by the support information derivation function 142 (step S140).

Next, the image generation function 145 generates an image including the support information for provision (step S150). Next, the display control function 146 displays the generated image on the display 130 in a predetermined display mode (step S160). Accordingly, processing of this flowchart ends.

As described above, according to the embodiment, in the medical information processing device 100, the processing circuitry 140 acquires medical information regarding a subject and a predetermined medical treatment support model. Further, the processing circuitry 140 inputs the medical information to the predetermined medical treatment support model to derive support information regarding a predetermined medical treatment. Further, the processing circuitry 140 derives a suitability between the clinical data and the predetermined medical treatment support model on the basis of the clinical data and the predetermined medical treatment support model. Further, the processing circuitry 140 displays the support information and the suitability on the display 130 in association with each other. As a result, it is possible to provide more appropriate support information to a user who examines the subject.

In addition, according to the embodiment, clinical data of a specific disease or medical department can be used by doctors of other specialized medical departments or general medical treatment according to a medical treatment support model, for example, and thus it is possible to early detect a disease that is difficult to determine. Further, according to the embodiment, more appropriate support information can be provided to a subject having an undetermined or unknown disease.

Further, according to the embodiment, it is possible to allow a user to ascertain a suitability and more appropriately determine whether to use support information depending on the suitability by displaying only support information with a high suitability, for example, in the case of disease risk. In the case of similar case search, it is possible to search for cases with higher similarities by deriving a suitability for each of the target subject and other subjects and searching for similar cases on the basis of suitabilities and similarities. Further, according to the embodiment, it is possible to allow the user to perform appropriate medical examination by predicting an additional examination item having a high suitability and presenting the predicted additional examination item to the user.

Any of the embodiments described above can be represented as follows.

A medical information processing device including:

a storage that stores a program; and

a processor is configured to, by executing the program:

acquire clinical data on a subject and a predetermined medical treatment support model;

input the clinical data to the predetermined medical treatment support model to derive support information regarding a predetermined medical treatment;

derive a suitability between the clinical data and the predetermined medical treatment support model on the basis of the clinical data and the predetermined medical treatment support model; and

display the support information and the suitability in association with each other.

Although several embodiments have been described, these embodiments are presented as examples and are not intended to limit the scope of the invention. These embodiments can be implemented in various other embodiments, and various omissions, replacements, and changes can be made without departing from the gist of the invention. These embodiments and modifications thereof are included in the scope of the invention described in the claims and the equivalent scope thereof, as are included in the scope and gist of the invention.

What is claimed is:

1. A medical information processing device comprising a processing circuitry configured to:
acquire clinical data on a subject and a predetermined medical treatment support model;
input the clinical data to the predetermined medical treatment support model to derive support information regarding a predetermined medical treatment;
derive a suitability between the clinical data and the predetermined medical treatment support model on the basis of the clinical data and the predetermined medical treatment support model; and
display the support information and the suitability in association with each other.

2. The medical information processing device according to claim 1, wherein the processing circuitry selects a medical treatment support model from a plurality of medical treatment support models on the basis of items included in the clinical data, and inputs the clinical data to the selected medical treatment support model to derive the support information.

3. The medical information processing device according to claim 1, wherein the processing circuitry selects, from the items included in the clinical data, input items to the medical treatment support model associated with a disease to be derived, and inputs information of the selected input items to the medical treatment support model to derive the support information.

4. The medical information processing device according to claim 1, wherein the processing circuitry derives the suitability on the basis of a distance or a likelihood between a feature vector based on the clinical data of the subject in a vector space and the medical treatment support model in the vector space.

5. The medical information processing device according to claim 4, wherein, when the medical treatment support model is an identification model, the processing circuitry derives a likelihood of the support information on the basis of an objective function corresponding to the medical treatment support model, the support information, and a pseudo classification label in the support information, and derives the suitability on the basis of the derived likelihood.

6. The medical information processing device according to claim 4, wherein, when there is a single feature vector, the processing circuitry derives the suitability on the basis of a distance between the feature vector and the medical treatment support model if the medical treatment support model is a linear generation model, and derives the suitability on the basis of a likelihood when the clinical data corresponding to the feature vector is applied to the medical treatment support model.

7. The medical information processing device according to claim 4, wherein, when there are a plurality of feature vectors, the processing circuitry derives the suitability on the basis of a distance between a subspace based on the plurality of feature vectors and the medical treatment support model when the medical treatment support model is a linear generation model, and when the medical treatment support model is a non-linear generation model, generates a probability distribution for the plurality of the feature vectors and derives the suitability on the basis of a distance of a provability distribution space in the generated probability distribution and a probability distribution corresponding to the medical treatment support model.

8. The medical information processing device according to claim 1, wherein the processing circuitry further generates an image to be displayed on a display, and the image includes an image showing support information having a derived suitability equal to or greater than a threshold value.

9. The medical information processing device according to claim 8, wherein the processing circuitry displays support information having a suitability equal to or greater than the threshold value and support information having a suitability less than the threshold value in a distinguishable manner.

10. The medical information processing device according to claim 1, wherein the processing circuitry displays support information of other subjects having a similarity equal to or greater than a threshold value to the support information of the subject.

11. The medical information processing device according to claim 1, wherein the processing circuitry displays information on an additional examination item that is not included in the clinical data and is predicted to have a high suitability.

12. A medical information processing method performed by a computer of a medical information processing device, the medical information processing method comprising:
acquiring clinical data on a subject and a predetermined medical treatment support model;
inputting the clinical data to the predetermined medical treatment support model to derive support information regarding a predetermined medical treatment;
deriving a suitability between the clinical data and the predetermined medical treatment support model on the basis of the clinical data and the predetermined medical treatment support model; and
displaying the support information and the suitability in association with each other.

13. A non-temporary computer-readable recording medium storing a program causing a computer of a medical information processing device to:
acquire clinical data on a subject and a predetermined medical treatment support model;
input the clinical data to the predetermined medical treatment support model to derive support information regarding a predetermined medical treatment;
derive a suitability between the clinical data and the predetermined medical treatment support model on the basis of the clinical data and the predetermined medical treatment support model; and
display the support information and the suitability in association with each other.
